(19) United States
(12) Patent Application Publication
(10) Pub. No.: US 2006/0048772 A1
(43) Pub. Date: Mar. 9, 2006

(54) Inhalation therapy device

(75) Inventor: Markus Borgschulte, Munchen (DE)

Correspondence Address: MERCHANT & GOULD PC, P.O. BOX 2903, MINNEAPOLIS, MN, 55402-0903, US

(73) Assignee: Pari GmbH Spezialisten fur effektive Inhalation, Starnberg (DE)

(21) Appl. No.: 11/196,220

(22) Filed: Aug. 3, 2005

(30) Foreign Application Priority Data
Aug. 27, 2004 (DE) 10 2004 041 667.2

(51) Int. Cl. A61M 11/00; B05B 17/06

(52) U.S. Cl. 128/200.14; 128/200.16

(57) ABSTRACT

An inhalation therapy device for providing a medicament in the form of an aerosol for inhalation, said device comprising a storage means for a medicament, which enables an improved deposition of medicament droplets in the respiratory tract of a patient. The inhalation therapy device comprises a first electric contact means which is in contact with the medicament and a second electric contact means which is in contact with the patient, wherein by applying a voltage it is possible to cause an electrostatic charge between the medicament and the patient, which leads to an improved deposition of the medicament in the respiratory tract of a patient.

### SUBJECT MATTER OF THE PRESENT INVENTION

The present invention relates to an inhalation therapy device for the provision of a medicament in the form of an aerosol for inhalation, and in particular to an inhalation therapy device which enables a more efficient and targeted use by a user of the inhalation therapy device.

### BACKGROUND OF THE PRESENT INVENTION

Numerous respiratory tract diseases can be treated by means of an inhalation therapy, and it is thus possible to cure the respective respiratory tract disease or at least alleviate the complaints.

In inhalation therapy, an aerosol containing droplets of medicament is provided, which is inhaled by the patient with the inhaled air and can thus exert its effect in the respiratory tract. The site at which the droplets of medicament contained in the aerosol are deposited in the respiratory tract depends, inter alia, on the size of the droplets. In an aerosol having comparatively large droplets, there is a high likelihood that the droplets will already be deposited in the upper respiratory tract, whereas in an aerosol with smaller droplets, the droplets will also reach the lower respiratory tract, for example to the alveoli of the lungs. However, if the droplet size is too small, this may result in the droplets of medicament not being deposited in the respiratory tract at all, but rather exiting the respiratory tract of the patient during exhalation without having any effect and being released into the environment. Owing to the anatomic features of the patient or to the type of nebulising device or aerosol generating device used, it is often not possible to exactly adjust the size of the droplets in order to apply the medicament droplets of the aerosol at precisely the desired site in the respiratory tract. Large losses can thus sometimes occur when using the aerosol, for example, if the droplets are too large, the droplets are already deposited in the upper regions of the respiratory tract even though they are not supposed to be applied until the lower regions of the respiratory tract, or if the droplets are too small, the droplets exit the respiratory tract again without having being applied at all.

A therapeutic problem which thereby arises is that it cannot be determined with certainty how much of the medicament in the generated aerosol could actually be applied at the desired site in the respiratory tract. A further problem is that some medicaments for generating an aerosol are very expensive, and it is thus desirable to achieve an effective use of the medicament and to prevent high losses of medicament as a result of non-occurring deposition. Furthermore, effects on the environment and third parties, which are caused by non-deposited droplets that are exhaled by the patient and are thus released into the ambient air, could also prove problematic.

The object of the present invention is to provide an inhalation therapy device in order to achieve an improvement in the deposition of droplets of medicament in the respiratory tract of a patient.

The object of the present invention is solved by an inhalation therapy device for providing a medicament in the form of an aerosol for inhalation, said device having a storage means for the medicament, a first electric contact means which is conductively in contact with a medicament present in the storage means, a second electric contact means which is in contact with a user of the inhalation therapy device during use of the same, a voltage supply means having a first and a second connecting device, said first connecting device being electrically connected with one of the first or the second electric contact means, and a switching device which can be switched such that either in a first position of the switching device, the other of the first or the second electric contact means is electrically connected with the first electric contact means or the second electric contact means is electrically connected with the second connecting device of the voltage supply means.

It can hereby be achieved that the medicament droplets of the aerosol are electrostatically charged by applying an electric potential. By applying an electric field, movement of the electrostatically charged droplets of the aerosol can be controlled. Since a predetermined electric potential is also applied to the user or patient, it is possible to make the user either attract or repel the electrostatically charged droplets of the aerosol. This behaviour can be used to increase deposition of the droplets of medicament at the desired sites in the lungs.

The inhalation therapy device advantageously also comprises a control means for controlling the switching device. The inhalation therapy device furthermore advantageously comprises a detection device for detecting the respiratory flow. The detection device is advantageously designed such that it supplies detection signals to the control means, on the basis of which the switching device can be controlled.

The use of a detection device makes it possible to process information regarding the location and time of the aerosol in the inhalation therapy device.

The voltage supply means is advantageously a diode cascade. The second electric contact means is advantageously provided on a mouthpiece of the inhalation therapy device.

It can thereby be advantageously achieved that the patient almost unavoidably comes into contact with the electric contact means such that a secure function of the device according to the invention can be ensured.

If there is a flow essentially from the inhalation therapy device to the user, the switching device is advantageously in the first position. Furthermore, if there is a flow essentially from the user to the inhalation therapy device, the switching device is advantageously in the second position.

It can thus be advantageously ensured that the electrostatically charged droplets of the aerosol have the same electric potential as the patient during inhalation, and thus an unintended deposition of the droplets contained in the aerosol owing to an electrostatic force acting on the aerosol can be prevented. During exhalation, the patient is advantageously brought to a different electrostatic potential so that a potential gradient occurs between the electrostatically charged droplets of the aerosol and the patient, owing to which electrostatic forces act on the droplets of the aerosol, thus benefiting deposition of the droplets of the aerosol in the respiratory tract owing to this force effect.

### DESCRIPTION OF THE FIGURE

FIG. 1 of the present invention shows a circuit diagram and a schematic design of the inhalation therapy device according to the present invention.

FIG. 1 shows an inhalation therapy device 1, which, in the embodiment shown herein, consists of a nebulising unit 10 and a control unit 20. The nebulising unit comprises a nebulising chamber 15 in or on which a nebulising means 13 is disposed, which is in contact with a medicament 12, for example in the form of a liquid, which is disposed in a storage means 11, for example in the form of a medicament tank, and which nebulises said medicament 12 during operation of the inhalation therapy device such that an aerosol 14 is created which is released into the nebulising chamber 15. The patient can inhale this aerosol 14 via a mouthpiece 16.

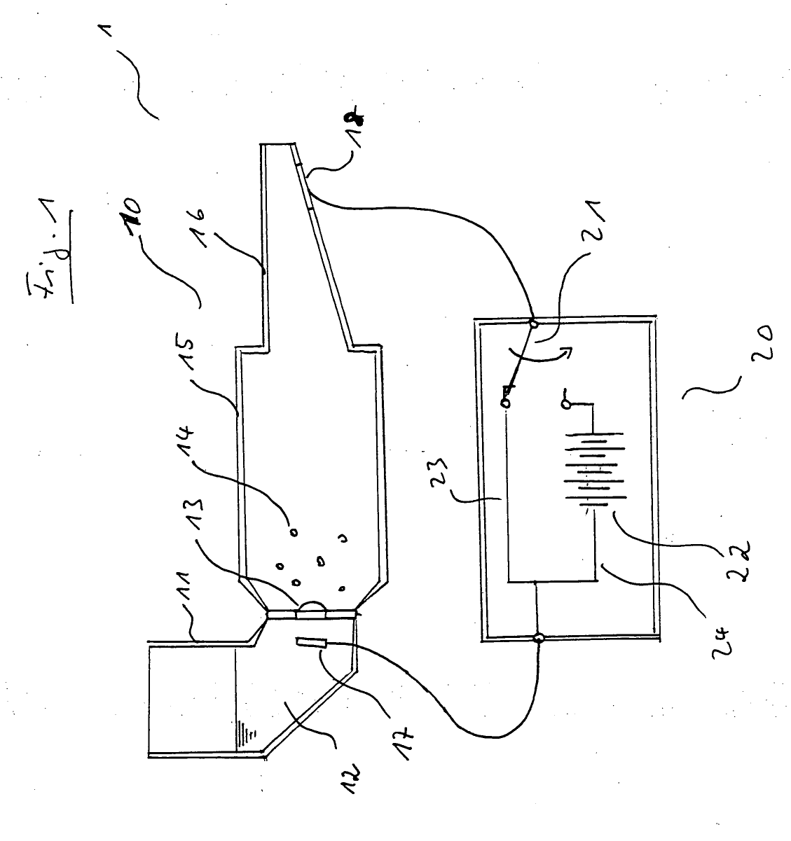

An electric contact means 17, for example in the form of an electrode, which is in contact with the medicament 12 in an electrically conductive manner and is connected with the control unit 20, is furthermore disposed in or on the storage means 11. A further electric contact means 18, for example in the form of an electrode, is disposed on the mouthpiece 16 and is also connected with the control unit 20. A patient using the nebulising unit comes almost unavoidably into contact with the electrode 18 and the electrode 18 is thus generally in conductive contact with the patient. The electrode 17 can also be advantageously integrated in the nebulising means 13 such that no further elements are necessary, in particular if the nebulising means also contains metal parts which ensure an electrically conductive connection with the medicament 12 in the storage means 11. According to the shown embodiment, a switching device 21, for example in the form of a change-over switch, is disposed in the control unit 20, which either connects the electrode 18 directly with the electrode 17 in an electrically conductive manner via the upper path 23 such that there is no potential difference between the electrode 17 and the electrode 18, or which connects the electrode 18 via the voltage supply means 22 using the lower path 24 such that there is a potential difference between the electrode 17 and the electrode 18 to the amount of the voltage or potential difference provided by the voltage supply means. The switching device 21 can, as regards electrics, be connected both with the electrode 18 on the side of the mouthpiece, as shown in FIG. 1, and with the electrode 17 on the side of the medicament (not shown here).

A detection device, for example in the form of a so-called flow sensor, can furthermore be provided, which detects whether there is an inhalation flow or an exhalation flow. This detection device is not shown in FIG. 1. The detection device can be provided as a separate device or can be integrated in the nebulising means 13.

Moreover, further inhalation and exhalation valves can be provided on the mouthpiece or in or on the nebulising chamber (not shown here), which make it possible to conduct and control a respiratory flow inside the nebulising chamber and the mouthpiece. The nebulising means can furthermore be electrically controlled (also not shown). The nebulising unit 10 and the control unit 20 can furthermore be accommodated in one single housing, however also be mechanically separable in order to ensure possible cleaning of the nebulising unit 10.

The electrode 17 or an electrode integrated in the nebulising means 13 is connected in an electrically conductive manner with the medicament such that the medicament droplets in the aerosol can be electrostatically charged. During inhalation, the user is charged with the same potential in that the switching device 21 connects the electrode 17 with the electrode 18 via the upper path 23. At the end of inhalation or when the aerosol is in the target region in the lungs, the desired potential difference between the aerosol and the user is switched. The switching device 21 is thereby located in the lower position such that it connects the electrode 17 with the electrode 18 via the voltage supply means 22 using the lower path 24. A potential gradient results owing to the potential difference, thereby causing electrostatic forces to occur between the electrically charged droplets of the aerosol and the differently charged patient. The electrostatic forces lead to an attraction force between the patient and the droplets of the aerosol, and this benefits aerosol deposition at the respective site. Exhalation losses, which would otherwise occur due to exhalation of the medicament aerosol if deposition of the medicament did not take place, are thus reduced. Since there is no potential difference between the aerosol and the patient during inhalation, there is no deposition in the upper respiratory tract owing to differences in charge.

The nebulising means or parts thereof can advantageously form a pole of the voltage supply means 22, for example in the form of a high-voltage source. A so-called user pole in the form of the electrode 18 is set to the potential of the electrode 17 during inhalation, with the user pole in the form of the electrode 18 being connected to the other pole of the voltage supply means 22 during exhalation, the first pole not being connected with either of the two electrodes 17 and 18.

1. An inhalation therapy device for the provision of a medicament in the form of an aerosol for inhalation, comprising
a storage means for the medicament,
a first electric contact means conductively in contact with the medicament disposed in the storage means,
a second electric contact means in contact with a user of the inhalation therapy device during use of the same,
a voltage supply means comprising a first and a second connecting device, said first connecting device being electrically connected with one of the first or the second electric contact means, and
a switching device which can be switched such that in a first position of the switching device, the second electric contact means is electrically connected with the first electric contact means, and in a second position of the switching device, the other of the first or the second electric contact means is electrically connected with the second connecting device of the voltage supply means.

2. An inhalation therapy device according to claim 1, comprising control means for controlling the switching device.

3. An inhalation therapy device according to claim 1, comprising a detection device for detecting the respiratory flow.

4. An inhalation therapy device according to claim 3, wherein the detection device is configured to supply detection signals to the control means, on the basis of which the switching device can be controlled.

5. An inhalation therapy device according to claim 1 wherein the voltage supply means comprise a diode cascade.

6. An inhalation therapy device according to claim 1 wherein the second electric contact means is provided on a mouthpiece of the inhalation therapy device.

7. An inhalation therapy device according to claim 1 wherein the switching device is in the first position when there is a flow essentially from the inhalation therapy device to the user.

8. An inhalation therapy device according to claim 1 wherein the switching device is in the second position when there is a flow essentially from the user to the inhalation therapy device.
